(19) United States
(12) Patent Application Publication
(10) Pub. No.: US 2003/0119201 A1
(43) Pub. Date: Jun. 26, 2003

(54) Method for determination of product and substrate concentrations in a medium

(76) Inventors: Evelyn Wolfram, Julich (DE); Matthias Arnold, Weilerswist (DE); Andreas Franz, Wesseling (DE); Dirk Weuster-Botz, Dachau (DE); Christian Wandrey, Julich (DE)

Correspondence Address: Joseph W. Berenato, III, Liniak, Berenato & White, LLC, 6550 Rock Spring Drive, Suite 240, Bethesda, MD, 20817, US

(21) Appl. No.: 10/301,851

(22) Filed: Nov. 22, 2002

Related U.S. Application Data
(63) Continuation of application No. PCT/EP01/05891, filed on Nov. 29, 2001.

(51) Int. Cl. G01N 33/00

(52) U.S. Cl. 436/178; 73/61.59; 73/1.02

(57) ABSTRACT

A method for determining substrate and product concentration in liquid and/or gaseous media is disclosed. Several samples of at least one substance to be analyzed are removed in at least one sampling region by time-controlled diffusion of the analyte between the medium and a diffusion medium, which is fed to the sampling regions through fluid conduits using at least one pump and semipermeable membranes. The diffusion medium is transported to at least one detector while simultaneously new diffusion medium is fed from the sampling region, and analyzed to determine the analyte concentration. The detector provides a temporal concentration distribution or a temporal distribution of a signal proportional to the concentration. A change in the ratio of the signal maximum to the base line is ascertained in the output of the detector signal and, on the basis of this, a change in the diffusion properties of the semipermeable membrane is inferred and a correction factor ascertained.

CROSS-REFERENCE TO RELATED APPLICATIONS

This application is a continuation of pending PCT application no. PCT/EP01/05891, the disclosure of which is incorporated herein by reference. A claim to priority under 35 U.S.C. §120 is hereby made to PCT/EP01/05891.

### FIELD OF THE INVENTION

The present invention concerns a method for determining substrate and product concentrations in liquid and/or gaseous media in which several samples are taken of at least one substance to be analyzed-the analyte-in at least one sampling region by time-controlled diffusion of the at least one analyte between the respective medium and a diffusion medium which is fed to the sampling region by fluid conduits using a pump through semipermeable membranes. Subsequently the diffusion medium is transported from the sampling region to at least one detector to which a new diffusion medium is fed in at the same time, and is analyzed by this to determine the analyte concentration. Furthermore the invention concerns a device for implementing the method.

### BACKGROUND OF THE INVENTION

In various regions of natural and engineering science, especially in biology and chemistry as well as in biological and chemical process and environmental engineering, it is necessary and desirable to measure the concentration of certain substances in a large number of reaction mixtures at the same time, whereby in particular a particular significance accrues to online analysis.

Known approaches in this connection consist in allocating a sensor arrangement with a complete measuring region to one reaction container each whereby sensors are used which can be introduced directly in a fluid stream escaping from this. Here the precondition is that the sensor can come into contact with the medium in which the concentration of a substance is to be measured, that is, it is not directly attacked by the medium. Furthermore the limiting conditions, for example, the pH value and temperature, permit direct application and a sufficiently exact measurement in the undiluted medium. Many sensors do not meet these technical preconditions. For example, with sensors for immobilized enzymes, the instability of the enzyme, above all at higher temperatures (steam sterilization), and the restricted measurement range prevent direct application. For these reasons, the detectors are arranged outside the reactor container in known online analysis apparatus. Sampling takes place here regularly in that volumes of medium are removed from reaction containers to be sampled and fed to an analysis device or detector through transport conduits. Frequent volume sampling is nonetheless possible only with containers in which the volume removed is very small in relation to the reaction volume. That means that with this sampling strategy, the frequencies and extent of sampling depend upon the reaction volume and are directly restricted by it.

For this reason, conducting sampling by time-controlled diffusion of the analyte from the medium to be sampled into an acceptor liquid through dialysis tubes is proposed in DE 197 29 492 A1. Moreover, the enrichment of the analyte into the diffusion medium and therewith the sampling is controlled through diffusion time. This procedure has the advantage that only molecules are removed from the medium, but no volume of medium. Sampling is thus limited only by the overall amount of substance and not by the reaction volume.

With the known methods, the transport of the acceptor fluid through the facility takes place using a pump. This is turned off after filling the dialysis tubes with fresh acceptor fluid so that analytes that are present in the reaction range in higher concentrations are accommodated by diffusion into the acceptor fluid through the walling of the dialysis tube.

The sample is then fed to a suitable detector for analysis. The problem with this procedure is that the diffusion properties of the membrane can change, for example as a consequence of deposits (fouling effect), owing to which the measurement results are falsified.

### SUMMARY OF THE INVENTION

The object of the invention is therefore to refine a method of the type mentioned at the beginning such that with comparatively little expenditure, impairments of the diffusion properties can be taken into consideration in the measurements.

This object is accomplished in accordance with the invention in that the detector provides a temporal concentration distribution or a temporal distribution of a signal proportional to the concentration, whereby then an inference is made through a calibration and a corresponding evaluation of the detector signals as to the analyte concentration in the sampled medium, and in that a change in the ratio of the signal maximum to the base line is ascertained in the output of the detector signal, and a change in the diffusion properties of the semipermeable membranes is inferred on the basis of this and a corresponding correction factor is ascertained and taken into consideration in the further issue. In this way, a possible drift owing to a change of diffusion properties, for example on the basis of blockages or coatings (fouling) can be taken into consideration through the data evaluation.

Alternatively, or in addition, it is possible to ascertain two signals at different flow rates of the diffusion medium and/or different diffusion times with a resting medium in close temporal succession and to compare them with one another with respect to their characteristic properties in order to recognize on this basis a possible drift due to fouling which then can correspondingly be considered in evaluating the data. In addition, in this case a temporal change in analyte concentration known on the basis of several measurements and/or a dynamic model can be taken into consideration.

It is provided in accordance with a further aspect of the present invention that a bypass conduit is provided through which diffusion medium is guided by the pump past the sampling region to the detector. This bypass can likewise be actuated through a multivalve or multipath valve arrangement and alternatively to the sampling regions. Diffusion medium, for example, can be conducted due to the presence of such a bypass conduit if in the meantime at one time; no sampling region is to be flowed through. It is also possible to inject a standard medium into the diffusion medium in the region of the bypass and to transport this segment by connecting the bypass to the detector. By conducting this process repeatedly before and during the duration of the test, drift phenomena of the detector can be corrected.

In accordance with one embodiment, it is provided that several sampling regions are provided according to the manner of a parallel connection. The at least one pump operates continuously and a provided multivalve or multipath valve arrangement connected in front of the sampling region in the conduit for the diffusion medium is controlled such that in the change in any given case diffusion medium flows through one sampling region a transport in the remaining sampling regions is eliminated, whereby preferably the valve arrangement is controlled such that basically diffusion medium continuously flows in turn through one of the parallel fluid conduit regions to the detector in any given case.

As is the processes known on the basis of DE 197 29 492 A1, sampling is consequently subdivided in the individual sampling regions into a first segment in which the diffusion medium is at rest and a diffusion of the analyte between the medium to be sampled and the diffusion medium takes place, and a second segment in which the diffusion medium is transported from the sampling region to the detector and is analyzed in the through flow with respect to the concentration of analyte. In contrast to the known method in which transport is conducted through an intermittently operating pump, with the method of the invention, a continuously operating pump is now used so that a transport of the acceptor from a sampling region to the detector can take place simply, since that fluid conduit segment which contains the sampling region is connected through appropriate actuation of the multipath or multivalve arrangement with the pump. Since the valves can be actuated very exactly, the opening and closing processes can be conducted in time-optimized manner. An actuation of the pump is not necessary at all.

The use of a continuously operating pump furthermore has the advantage that the valve arrangement can be so actuated that a transport of diffusion medium connected with simultaneous analysis in the detector basically takes place continuously in one of the sampling regions while at the same time sampling takes place in the other sampling regions by diffusion. Consequently the possibility of basically undertaking continuous analyses is opened up. That was not possible with the known methods, at least during standstill times of the pump.

An especially efficient sampling is attained if in parallel sampling regions in any given case the diffusion or sampling time of a region is at least the measuring time necessary for signal recording in the detector of all other parallel sampling regions together. The diffusion times are correspondingly adjusted to one another such that during sampling in one region, the measurements for the other sampling regions can be undertaken simultaneously one after the other and then the measurement of the sample can then also be directly joined to the diffusion. In this way, an especially high effectiveness and flexibility is attained.

It is provided in developing the invention that a pressure measurement takes place in series connected in front of the sampling regions in the conduit for the diffusion medium for recognition of a disturbance in a conduit segment. Underlying this is the consideration that, for example, when a leakage occurs in the conduits between the pump and the detector, a portion of the diffusion medium is not conducted through the detector, but rather into the defective conduit to the extent that conduit resistance is less in this direction than toward the detector. Sampling would thus not only be impaired in the defective region, but also in the overall system. Building in a pressure sensor makes possible here automatic disturbance recognition since the conduit pressure in connection with through flow of parallel regions moves in a value range characteristic for the device. If the pressure sinks outside the characteristic value range during flow through of one of the parallel conduit regions, a disturbance is present, namely in the event of an excessively low pressure, a leak, and in the event of an excessively high pressure, a stopping up, and the defective conduit region can be uncoupled.

In addition, a check valve can be connected after the sampling regions in series, or alternatively an additional multipath or multivalve arrangement can be provided which prevents a diffusion medium from flowing back from a sampling region into another sampling region.

Several detectors can be provided connected in series in an inherently familiar manner for simultaneous analysis of different analytes. Since according to experience, the detectors can also fail or sharply drift, it can also be appropriate to provide several detectors for the same analytes in parallel regions that can be turned on as a replacement in the event a detector fails. Electively, however, various detectors can also be connected in parallel through a multipath or multivalve valve arrangement, owing to which the possibility is opened of determining different analytes at various points in time. Such an interconnection, for example, is appropriate with detectors that mutually influence one another in their measuring processes.

In accordance with a further aspect of the invention, the device contains a sample preparation module connected in series in front of the detector which either absorbs disturbing components from the diffusion medium (for example, activated charcoal) or transforms them reactively into a non-disturbing chemical form. Alternatively or additionally, there are also detectors that require a sample preparation module so that the analyte is transformed into a detectable form (for example, enzyme or dye reactions and the photometric measuring method).

In a preferred manner, a diffusion medium is used which is basically free from the analytes to be detected so that the concentration gradient is high over the semipermeable membrane from the medium to be sampled to the diffusion medium. In cases in which the analyte concentration in the medium to be sampled falls below the detection limit of a detector, it can, however, also be appropriate to use a diffusion medium that contains a known concentration of the analyte or analytes that lies above the low concentration in the medium. Then a diffusion of the analyte into the medium takes place in the region of the sampling region and the loss in concentration over diffusion time is measured in the diffusion medium and used for determining the analyte concentration in the medium to be sampled.

The diffusion medium can be eliminated before undertaking the analysis. Alternatively, it is also possible to collect the samples in an automatic fraction collector for subsequent off-line analysis.

The components of a sample are quantified by being fed to appropriate detectors. Since is a matter of a relative measuring method in measuring the diffusively obtained sample segments, the measurement signals from unknown concentrations can only be ascertained in comparison with a standard mixture sampled through diffusion under operating conditions. Furnishing a standard solution into which a further semipermeable membrane is dipped separately from the other sampling sites, as this is known on the basis of DE 197 29 492 A1, does not suffice for such a calibration if the semipermeable membranes selected do not have exactly identical properties, such as, for example, the same length, surface and wall thickness. Empirically, such tubes are not manufactured so exactly in relation to these features with the consequence that a signal measured in the detector of a sample diffusively enriched in this manner cannot be relied upon for calibration. In accordance with the present invention, it is therefore provided that, for calibration, the semipermeable membranes are dipped in media of known analyte concentration, and in each case measurement data sets are compiled on the basis of which the measurement results furnished by the detector are evaluated for determining the analyte concentration.

Especially with sterile technique requirements, standard concentrations can also be directly deposited in the reaction containers. This prevents frequent medium changes and expensive sterilization measures in the containers. For this, known concentrations of at least one analyte are deposited into the preferably analyte-free medium through the addition of correspondingly calculated volumes of a concentrated standard mixture of the analyte. Then the reaction containers are sampled in the manner described above and corresponding measurement data sets are compiled. A renewed additional dosing of standard mixture into the reaction medium and subsequent measurement can be repeated until the highest concentration of analytes desired by the user is reached. In this way, the measurement range of analyte to be expected can be covered during the experiment.

The detector used can already be so adjusted internally or precalibrated that it directly determines the analyte concentrations in the samples passed through which are obtained through diffusion in the sampling regions. That is, the device supplies without further recalculation the analyte concentration present in the diffusion medium. On the basis of these analyte concentrations and the calibration method previously described, inferences can be made on the basis of these concentrations in the diffusion medium about the concentrations in the sampled medium with corresponding calculation models.

Furthermore, rinsing fluid can be fed to the detector through the bypass conduit.

With respect to further advantageous refinements of the present invention, reference is made to the dependent claims as well as to the following description of an embodiment of a device with which the process of the invention can be conducted on the basis of the drawing.

### DESCRIPTION OF THE FIGURES

FIG. 1 is a schematic view of a device according to the invention;

FIG. 2 is a graph illustrating a typical equilibration function in diffusion processes;

FIG. 3 is a graph illustrating a typical peak of a detector;

FIG. 4 is a graph illustrating convergence according to the invention.

### DETAILED DESCRIPTION OF THE PREFERRED EMBODIMENT(S)

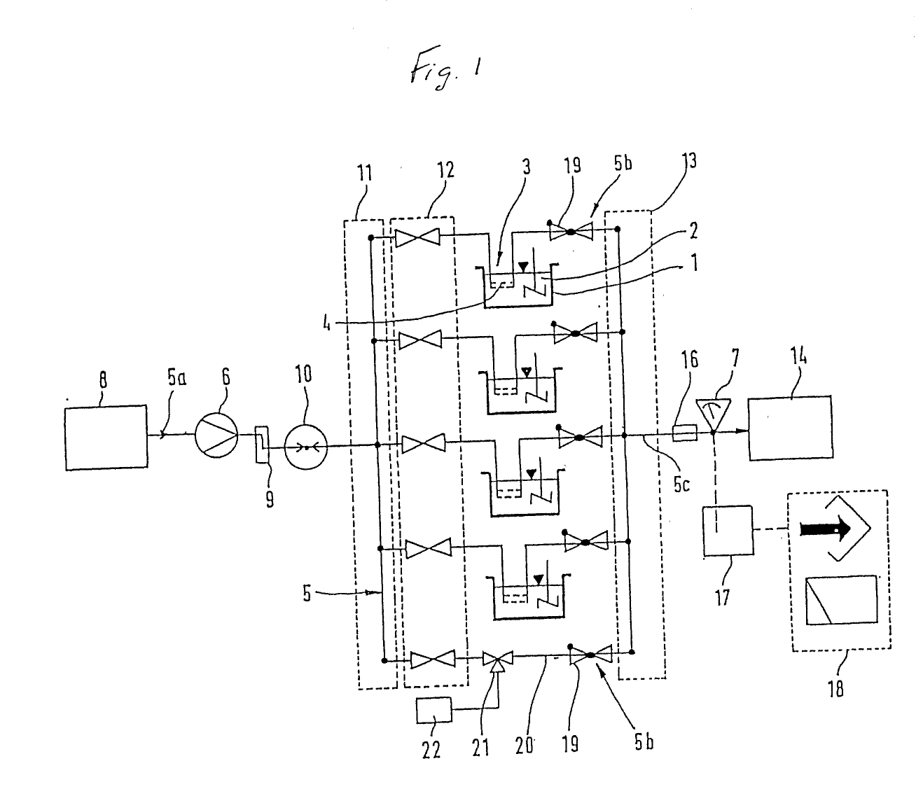

A device for determining substrate and production concentrations in liquid and/or gaseous media 2 according to the invention is best shown in FIG. 1. The device has a large number of reaction containers 1 in which in any given case a gaseous or liquid medium 2 to be analyzed is contained. With the reaction containers 1, it can, for example, be a matter of vibration cylinders that are kept constantly in motion. Through analysis, the concentrations of substances, of extracts or of reaction products, hereinafter called analytes, are measured inside the medium.

At least one sample module 3 is set in each reaction container 1 which has a semipermeable membrane 4 that is constructed in the form of a dialysis tube and is dipped completely into the medium 2 contained in the reaction container 1. The dialysis tubes 4 are arranged in the manner of a parallel connection and connected inlet-side through fluid conduits 5 with a pump 6 and outlet-side with a detector 7. The pump 6 is connected with a storage container 8 for accommodating a diffusion medium suited for a diffusion sampling, which can be gaseous or liquid as a function of the physical condition of the medium 2 to be sampled. A bubble trap 9 is provided arranged after the pump 6, which serves to remove bubbles from liquid diffusion medium. Moreover, a pressure sensor 10 is provided which measures conduit pressure.

The fluid conduit segment 5a coming from the pump 3 opens into a medium distributor 11 to which the parallel fluid conduit segments 5b are connected outlet side with sampling module 3, and a multivalve arrangement 12 is provided between the media distributor 11 and the sampling module 3 through which the parallel fluid conduit segments 5b are opened in each case for flow through of diffusion medium or can be closed for preventing such through flow.

On the outlet side, the sampling modules 3 open through the parallel fluid conduit segments 5b into a media collection module 13 which has on its outlet side a discharge 5c which leads to a detector 7 and an outflow lying behind it into a suitable waste reservoir 14 or into another type of drain for the diffusion medium. In the outflow 5c, a sampling preparation module 16 is provided before the detector viewed in the direction of flow, which absorbs disturbing components from the diffusion medium or reactively transforms them into a non-disturbing chemical form. Alternatively or additionally the sample preparation module 16 can also serve to transform the analyte into a form that can be recorded by the detector 7.

The signal output of the detector 7 is connected with a computer 18 through a measurement amplifier 17 that evaluates the measurement signals originating from the detector 7 and moreover also controls the valves of the multivalve arrangement 12 as well as the rate of conveyance of the pump 6.

Moreover stop valves 19 are provided in the fluid conduit segments 5b between the sampling modules 3 and the media collecting module 13 which in the event of a leakage in a fluid conduit segment 5b are supposed to prevent the diffusion medium which comes from a sampling module 3 from flowing into the defective conduit segment 5b instead of toward the detector 7.

In addition to the parallel sampling regions with the sampling modules 3 provided therein, a bypass conduit 20 is connected through a further valve of the multivalve arrangement 12 through which diffusion medium can be guided from the pump 6 past the sampling regions 5b to the detector 7. In this way, for example, the baseline of the detector 7 can be ascertained when fresh diffusion medium flows through or the detector 7 is rinsed with a rinsing agent through, for example, a pump connected only to the bypass 20. In addition, in the bypass, the possibility of introducing a sample segment of a standard mixture that is contained in a storage container 22 into the flow of the diffusion medium is provided through a three/two way valve 21 or another type of injection valve.

Parallel sampling with the device of the invention takes place as described below:

First of all, a suitable diffusion medium is pumped into the facility with the aid of the pump 6 until the fluid conduits 5 as well as the dialysis tubes 4 are completely filled with the diffusion medium. Proceeding from this setting, a sampling takes place in each case in the sampling modules 3 since with a continuously operating pump 6, the valve of the multivalve arrangement 12 connected in series in front of the corresponding fluid conduit region 5b is closed so that the diffusion medium rests in the sampling module 3 of this fluid conduit region 5b. This condition is maintained during a specified duration so that by diffusion, an adaptation of the concentrations of the analyte in the medium to be sampled, which is contained in reaction container 1 and the diffusion medium, takes place. If one proceeds from the assumption that the analyte concentration in the medium to be sampled is higher than in the diffusion medium, an amount of analyte characteristic for the concentration of the analyte in the medium accumulates in the diffusion medium within the specified time period. If the analyte concentration is higher in the diffusion medium, analyte enrichment takes place in a reversed manner through the diffusion taking place. Moreover, it is advantageously assured in contrast to filtration that the volume of the medium contained in the reaction container 1 basically remains unchanged.

After a lapse of the specified period of time, the valve of this fluid conduit region 5b is opened once again so that the diffusion medium contained in the dialysis tube 4 enriched or depleted with analyte is transported to the detector 7, and at the same time new diffusion medium flows back into the fluid conduit region 5b. The sample segment is analyzed when it flows through the detector 7, whereby the detector 7 emits measurement signals to the computer that correspond to the respective concentrations of analyte in the allocated reaction containers 1. In what way the evaluation takes place is yet to be explained below.

A sampling can be undertaken in the previously described manner in all sampling modules 3 by diffusion between the medium 2 contained in the respective reaction container 1 and the diffusion medium and an analysis can subsequently be undertaken since the segment of the diffusion medium which is subjected to diffusion in the sampling module 3 is transported to the detector 7 and analyzed by this when it flows through. The analysis of the sample segments received in the individual sampling regions takes place in alternation one after the other, that is, time-shifted. In order to be able to conduct measurements nonstop, that is, continuously with the least loss times possible, the measurement times are in each case determined such that the measurement or transport time in one of the parallel fluid conduit segments 5b is equal to the sum of the diffusion times of the other parallel regions, or, conversely, the diffusion and sampling time of one region is at least the measuring time necessary for signal recording in the detector for all other parallel sampling regions together. In other words, the diffusion times on the one hand and the measurement times on the other are so harmonized with one another that, with the exception of some connection-conditioned delays, one of the parallel fluid conduit segments 5b is flowed through, and correspondingly the segment of diffusion medium which was previously subjected to diffusion is analyzed.

The detector can already be adjusted internally or pre-calibrated such that it directly determines the analyte concentrations in the samples passed through the sampling modules 3. That is, it provides the analyte concentration present in the diffusion medium without further recalculation. On the basis of these analyte concentrations, it is possible to infer the analyte concentration contained in the sampled medium by calculation on the basis of measurement series, which were obtained in the framework of a previously conducted calibration.

Alternatively, the detector can provide a temporal distribution of the concentration of sample flowing through (dwelling time curve) or a temporal distribution of a signal proportional to the concentration. On the basis of measured value series which were obtained in a previously conducted calibration process, an inference as to the analyte concentration in the sampled medium can be ascertained whereby various properties can be adduced for the evaluation, such as, for example, the peak maximum, an increase of the front face, the area under the curve, the base line in the downflow of the curve, etc. Since such analysis methods are basically known, they will not be gone into in detail here. Only for the sake of completeness is reference made in this regard to the content of the disclosure of DE 197 29 492 A1.

As already mentioned, the analyte concentration in the diffusion medium is measured and an inference is subsequently made on the unknown analyte concentration in the sampled medium 2. Since here it is a mater of a relative measurement process, a precalibration must take place in which the analyte concentration in the diffusion medium is placed in relation with the analyte concentration in the medium to be sampled 2.

Before conducting the series of tests, each sampling module 3 is dipped in at least one medium with known analyte concentration for this. With the same diffusion times and other adjustments of a device as in the planned experiment, the measurement is now conducted in each connected sampling module 3. In this way, a set of measurement data for each analyte is allocated to each sampling module 3. The connection so obtained between the concentration in the reaction container to be sampled and the detector response during transport of the sample obtained by diffusion through the detector is used for evaluating the computer signals obtained online in the experiment.

The precalibration can also be undertaken directly in the reaction container 1, since there a specific volume of a concentrated standard analyte mixture of known construction (which preferably is mixed with the medium to be sampled in order to prevent dilution of other components of the medium) is dosed in. Thus a concentration known through the dosing in arises. Then the reaction containers 1 are sampled in the manner described above and the measurement data are recorded. A renewed dosing in of the standard analyte mixture into the medium to be sampled and subsequent measurement can be repeated so often until the highest concentration of the analyte desired by the user is reached. Thus the expected measurement range of the analyte can be covered during the experiment.

In addition to the precalibration, an intermediate calibration can take place through the bypass 20 while the experiment is running. Alternatively, a sampling module is arranged in a bypass or in a further parallel region can be dipped in a standard mixture during the experiment and can be sampled at regular intervals for recalibration with the same diffusion time.

If a leak occurs during the time of the experiment in the fluid conduits 5 or the sampling modules 3, the diffusion medium could be passed through the medium collection module 13 from other sampling regions 5b not through the detector 7, but rather into the defective region insofar as the conduction resistance is less in this direction than in detector 7. Sampling would thus be impaired not only in the defective fluid conduit region 5b, but also in the entire system. The stop valves 19 or a multivalve arrangement to be used as an option arranged in the device prevent this.

With a liquid diffusion medium, the gauge in the medium to be sampled will rise in the event of a leak inside reaction container 1, and the medium is necessarily contaminated in this case. With a gaseous diffusion medium, the pressure can rise in a closed container 1.

With leaks outside the reaction container 1, recognition of the leak is obvious with liquids, but not with gases, for example. Disturbances in the conduit can be recognized by incorporating a pressure sensor 10. This is based upon the consideration that the conductance pressure in the parallel fluid conduit regions 5b moves in a value range characteristic for the device when parallel regions and the bypass 20 are flowed through. If the pressure lies outside these ranges when fluid flows through a conduit region 5b, there exists a disturbance and the defective region 5b can then be uncoupled, that is, no longer subject to through current. Concretely, when pressure is too low, there is a leak, and when it is too high, there is a stoppage.

If the diffusion properties of the dialysis tubes 4 or another semipermeable membrane worsen during the experiment, for example by coating of its surface with components from the medium to be sampled (fouling), then the concentration equalization will be less than without this coating with a specified sampling time (diffusion time) in the dialysis tubes 4. This error is recognizable by evaluating the detector signals and can be incorporated into the concentration calculation with the original calibration values as an online corrector. The signal which is recorded when a detector 7 is subjected to blow through is, for example, a peak which does not return to the base line level in the event that pure diffusion medium flows through. The signal approaches a level that arises when diffusion medium flows through the sampling module 3 through diffusion in connection with a through current (effect of a contact time-and therewith disturbance-dependent diffusion). In this way, the enrichment in diffusion medium is known at two different diffusion times. From the comparison of these two values and the change in their ratio to each other, the changes in diffusion properties, that is fouling, can be taken into consideration in the measurements and be corrected by calculation. The bases and an example for determining the current diffusion properties of a membrane will be described below:

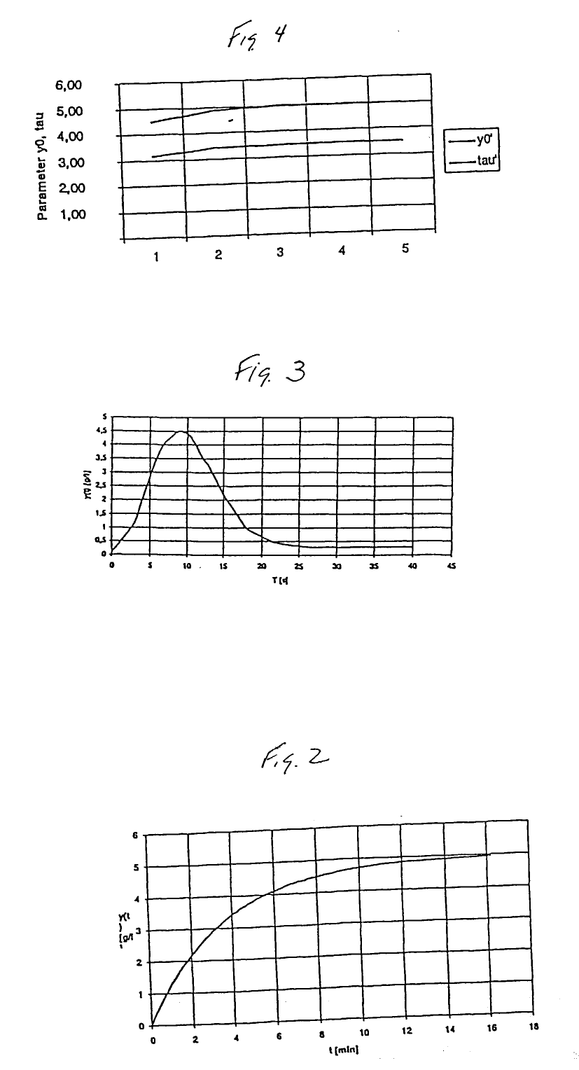

Equilibration processes such as the diffusion of analytes of a sample through a membrane into a diffusion medium considered here, are driven by concentration differences, as best shown in FIG. 2. For the following explanations, the initial concentration of the analytes in the diffusion medium will be set to zero solely for reasons of simplification. In addition, a possible baseline of the detector can be ascertained through the bypass arrangement and additively compensated for.

Let the concentration of the analyte in the sample be y₀. Let the sample volume be large in relation to the volume of diffusion medium. The typical course over time (step response) of the analyte concentration y in the diffusion medium is quantitatively described by the transition function represented in the following illustration.

FIG. 4 illustrates convergence according to the invention. In particular, y converges toward y₀ and the function is constant and monotonous. A change in the diffusion characteristics of the membrane in particular leads to a distortion (regioning or compression) of the function along the time axis. A rising diffusion resistance typically leads, for example, to slowing the equilibration process down and therewith to a regioning.

Generally such transition functions can be described by a trial solution:

y=f (y_{0,} τ, t)

whereby the parameter θ characterizes the temporal behavior of the function. The trial solution can be specialized for the diffusion processes regarded here to

y=y₀·(tτ)

y/y₀=g(tτ)

t/τ=g⁻¹(y/y₀)

The function g or its inverse function g⁻¹ qualitatively reproduce the curve of the normalized transition function. A change of the parameter τ leads to a regioning or compression of the function along the time axis.

If g and g⁻¹ are known, the parameters y₀ and τ can be determined iteratively with the method described here on the basis of successive measurement values with different contact times between sample and diffusion medium, as these in particular exist through a peak (peak value (resting dialysis) and subsequent saddle value (continuous dialysis), for example through the control computer of the device:

Input data

T₂: Contact time of longer measurement (for example, duration of the stop phase)

y₂: Allocated initial value of the detector (for example, peak maximum)

T₁: Contact time of shorter measurement (for example, T=V/F continuous dialysis)

y₁: Allocated initial value of the detector (for example following saddle value)

Beginning of iteration:

y₀⁽¹⁾=y₂

τ⁽¹⁾=T₁/g⁻¹(y₁/y₀⁽¹⁾)

Iteration:

y₀⁽¹⁾=y₂/g(T₂/τ^{(1-t)})

τ⁽¹⁾=T₁/g⁻¹(y₁/y₀⁽¹⁾)

Iteration interruption, for example under one of the conditions:

|y₀⁽¹⁾−y₀^{(1-tτ)}|≦E_{y}

|τ⁽¹⁾−τ^{(1-t)}|≦Eτ

i≧j

This process converges for transition functions with the above-described properties and supplies the true concentration value in the sample as well as with the parameter τ the current diffusion properties of the membrane online on the basis of measured values of an individual peak curve.

### EXAMPLE

The curve over time of the concentration change in the diffusion medium with dialysis is given through the following function

y=y₀·(1−e^{-1/τ)}

On the basis of this there result the iteration equations:

y₀⁽¹⁾=y₂/1−e^{-τ2}/τ^{(1-t)}

τ⁽¹⁾=T₁/−1n(1−y₁/y₀⁽¹⁾)

The (unknown) process values are

y₀=5 g/l current analyte concentration in the sample

τ=3.5 min current diffusion time constant of the membrane for the analyte

FIG. 3 illustrates a typical peak using the detector of the invention. The values ascertained on the basis of the current peak signal of the detector amount to:

T₂=8 min duration of the stop phase

y₂=4.49 g/l allocated initial value of the detector (peak maximum)

T₁=0.2 min contact time during continuous dialysis

y₁=0.278 g/l allocated initial value of the detector (saddle point)

A possible baseline is already ascertained as described above and taken into consideration (subtracted) in these values. Table 1 contains information about the method and what is supplied for the individual iteration steps.

TABLE 1

| Iteration step | y₀ | τ |
|---|---|---|
| 1: Start of iteration | 4.49 | 3.13 |
| 2 | 4.87 | 3.40 |
| 3 | 4.96 | 3.47 |
| 4 | 4.99 | 3.49 |
| 5 | 4.99 | 3.49 |

The current values for analyte concentration y₀ and time constant τ are approximated in a few steps by using the process described here. The convergence of the method is once again illustrated in FIG. 4. As already explained further above, the method converges not only for the example shown with a transition function given through an analytic expression, but also for all possible equilibration processes for which the function g(t) can be qualitatively indicated.

A single detector 7 is used in the device of FIG. 1. Since such a detector 7 can fail or drift sharply, optimally several detectors can be provided for the same analyte which can be electively turned on or turned off, for example in the event of the failure of a detector 7. Electively different detectors can also be connected parallel, for example through multipath or multivalve arrangements so that various analytes can be analyzed at various points in time. Such an interconnection is, for example, appropriate in detectors that mutually influence one another in their measuring process.

In sum, the previously described device operates in a very efficient manner since a measurement can take place practically continuously in the detector 7 provided, whereby when the pump 6 operates continuously, the individual parallel sampling regions 5b are opened for a transport of diffusion medium or closed during the diffusion time simply through actuation of the multivalve arrangement 12. Of course, it is also possible to use several pumps for the diffusion medium.

We claim:

1. A method for determining substrate and product concentration in liquid and/or gaseous media in which several samples of at least one substance to be analyzed-the analyte-are removed in at least one sampling region (3) by time-controlled diffusion of the at least one analyte between the respective medium and a diffusion medium which is fed to the sampling regions (3) through fluid conduits (5a, 5b) using at least one pump (6) by semipermeable membranes (2) and subsequently the diffusion medium is transported to at least one detector (7) while simultaneously new diffusion medium is being fed from the sampling region (3) and is analyzed by this to determine the analyte concentration, characterized in that the detector provides a temporal concentration distribution or a temporal distribution of a signal proportional to the concentration, in that a change in the ratio of the signal maximum to the base line is ascertained in the output of the detector signal, and on the basis of this, a change in the diffusion properties of the semipermeable membrane is inferred and a correction factor is ascertained.

2. Method according to claim 1, characterized in that an inference is made as to the analyte concentration in the sampled medium through a calibration of the detector (7) and a corresponding evaluation of detector signals, whereby the maximal rise of the front face of the detector signal, the signal maximum, the surface under the signal curve or the increased baseline following through flow of the peak maximum which results from the diffusion of the analyte into the diffusion medium are adduced for the evaluation.

3. Method according to claim 2, characterized in that several properties of detector signal distribution are used for evaluation at the same time, or ratios of these values toward one another are used.

4. Method according to claim 1, characterized in that two signals at different flow rates of the diffusion medium and/or different diffusion times with resting medium are ascertained in close temporal sequence and compared with one another with respect to their characteristic properties in order to recognize and to correct a possible drift due to change in the diffusion properties.

5. Method according to claim 4, characterized in that in addition a change in analyte concentration over time known on the basis of several measurements and/or a dynamic model is taken into consideration.

6. Method according to claim 1, characterized in that several sampling regions (3) are provided in the manner of a parallel connection and in that the at least one pump (6) operates continuously and a multivalve or multipath arrangement (12) connected in series upstream from the sampling regions (3) in the fluid conduit (5b) provided for the diffusion medium is controlled such that in any given case diffusion medium flows through one of the parallel fluid conduit regions (5b) to the detector (7) and a transport is prevented in the remaining sampling regions in alternation.

7. Method according to claim 6, characterized in that the valve arrangement (12) is controlled such that one of the fluid conduit regions (5b) is basically continuously subjected to through flow in alternation in any given case.

8. Method according to claim 6, characterized in that in the parallel sampling regions (3), the diffusion or sampling time of one region is at all times at least the measuring time of all other parallel sampling regions necessary for signal recording in the detector together.

9. Method according to claim 1, characterized in that a pressure measuring unit is connected in series upstream from the sampling regions (3) in the fluid conduit (5a) for recognizing a disturbance in a conduit segment.

10. Method according to claim 1, characterized in that air or gas bubbles are removed from fluid diffusion medium before reaching the sampling regions (3) using a bubble trap (9).

11. Method according to claim 1, characterized in that the multipath or multivalve arrangement (12) is controlled by a computer (18).

12. Method according to claim 1, characterized in that several parallel connected detectors are provided and diffusion medium coming from the sampling regions is fed to one of the detectors through a multipath or multivalve arrangement.

13. Method according to claim 1, characterized in that in a sample preparation module (16) connected upstream in series from the detector (7), at least one substance which can disturb the detector used is absorbed or reactively transformed into a non-disturbing chemical form.

14. Method according to claim 1, characterized in that in a sample preparation module (16), the analyte is reactively transformed into a form measurable by the detector (7).

15. Method according to claim 1, characterized in that a diffusion medium is used which is basically free from analytes to be detected.

16. Method according to claim 1, characterized in that a diffusion medium is used which contains a known concentration of at least one analyte which lies above the concentration in the medium to be sampled so that a diffusion of the analyte from the diffusion medium into the medium to be sampled takes place in the region of the sampling region.

17. Method according to claim 1, characterized in that the samples obtained from parallel sampling regions through diffusion are gathered with an automatic fraction collector in the output of the sampling region or of the detector for a subsequent off-line analysis.

18. Method according to claim 1, characterized in that, for calibration, the semipermeable membranes are dipped in media of known analyte concentration and measurement data sets are compiled on the basis of which the measured results supplied by the detector are evaluated for determining analyte concentration.

19. Method according to claim 1, characterized in that the semipermeable membranes (2) are dipped for calibration in at least one reaction container with the medium to be used in the experiment, and in that known concentrations of at least one analyte is set through addition of correspondingly calculated volumes of a concentrated standard mixture of at least one analyte and measured data sets are compiled for the various concentrations on the basis of which the measurement results supplied by the detector are evaluated for determining the analyte concentration.

20. Method according to claim 18, characterized in that the end concentration of the at least one analyte at the same time represents the desired start concentration in the experiment mixture.

21. Method according to claim 18, characterized in that the detector (7) issues a value for the concentration of the analyte in the diffusion medium, and an inference is made by calculation about the concentration in the medium at past diffusion times by comparing this measured value with the measured values which were ascertained by the calibration method according to claims 18 to 20 with a known analyte concentration.

22. Method according to claim 1, characterized in that diffusion medium is guided past the sampling regions (3) to the detector (7) through a bypass conduit (20).

23. Method according to claim 22, characterized in that in the region of the bypass conduit (20), a standard medium is injected into the diffusion medium and this segment is transported by connecting the bypass conduit (20) to the detector (7) in order to correct drift phenomena of the detector (intermediate calibration).

24. Method according to claim 22, characterized in that rinsing fluid is fed to the detector through the bypass conduit (20).
